(19) United States
(12) Patent Application Publication
(10) Pub. No.: US 2026/0180665 A1
(43) Pub. Date: Jun. 25, 2026

(54) ANTENNA ARRAY FOR AUTOMATED MEDICAMENT DELIVERY SYSTEMS AND DEVICES AND OTHER WEARABLE DEVICES

(71) Applicant: Insulet Corporation, Acton, MA (US)

(72) Inventors: Colan Ryan, Escondido, CA (US); Ehsan Hajisaeid, Windham, NH (US)

(21) Appl. No.: 19/430,591

(22) Filed: Dec. 23, 2025

Related U.S. Application Data
(60) Provisional application No. 63/738,613, filed on Dec. 24, 2024.

(51) Int. Cl. H04B 7/08; A61M 5/172; H01Q 3/36; H01Q 9/04; H01Q 21/06; H04B 17/20

(52) U.S. Cl. CPC H04B 7/0874; A61M 5/172; H01Q 3/36; H01Q 21/065; H04B 17/202; A61M 2205/3584; A61M 2209/01; H01Q 9/0421

(57) ABSTRACT

Methods and systems for controlling an antenna array of an automated medicament delivery device are disclosed. The method includes causing the antenna array to transition between multiple array configurations. The multiple array configurations are chosen from among configurations that vary at least one characteristic of a signal emitted or received by one or more antennas of the antenna array, configurations that vary an activation status of the plurality of antennas, and configurations that vary a combination of the at least one characteristic of a signal and an activation status of the plurality of antennas. The method also includes measuring a power of a received signal for each configuration of the multiple array configurations of the antenna array. The method further includes selecting an array configuration of the multiple array configurations based at least in part on results of the measuring and setting the antenna array to the array configuration selected.

### CROSS-REFERENCE TO RELATED APPLICATIONS

This application claims the benefit under 35 U.S.C. § 119(e) of U.S. Provisional Patent Application Ser. No. 63/738,613, filed Dec. 24, 2024, the disclosure of which is hereby incorporated herein in its entirety by this reference.

### TECHNICAL FIELD

This disclosure relates generally to automated medicament delivery administration. Some embodiments relate to an antenna array for automated medicament delivery systems and devices and other wearable devices.

### BACKGROUND

Automated medicament delivery systems and devices (AMD, e.g., Automated Insulin Delivery (AID) device, without limitation) are often used to administer medicaments from a reservoir of the AMD to the body of a patient via a cannula inserted into the body to treat medical conditions (e.g., Type 1 Diabetes, without limitation).

The AMD often communicates with other devices, such an analyte sensor and a controller (e.g., a handheld electronic computing device, such as a mobile device or dedicated handheld controller).

Reliable communications between the AMD and other devices may be challenging, given the optimization of packaging space within the AMD and the fixed location of the AMD on a user-body during use. This fixed location results in the antenna of the AMD being close to the surface of the skin of the user-body, which may result in performance degradation of the antenna. The fields from the antenna may extend into the lossy tissue of the user-body, which may lower the efficiency and gain of the antenna. This degradation may impede communication of the AMD with other devices. Further, the high permittivity of human tissue may affect the impedance matching of the antenna so that the strength of the signal between the antenna and radio chip is reduced, which may further reduce an effective range of the antenna. Any degradation and range limitations caused by the proximity of the antenna to the user-body may be apparent during both transmitting and receiving operations of the AMD.

Reliable communication between the AMD and other devices may also depend on an orientation of the AMD on the user-body as the orientation of the AMD may direct the power emitted by the antenna away from the other devices.

### BRIEF SUMMARY

In one or more illustrative embodiments, a method for controlling an antenna array of an automated medicament delivery device configured to administer medicament to a user-body is disclosed. The method includes causing the antenna array to transition between multiple array configurations. The antenna array includes a plurality of antennas. The multiple array configurations may be chosen from among configurations that vary at least one characteristic of a signal emitted or received by one or more antennas of the plurality of antennas, configurations that vary an activation status of the plurality of antennas, and configurations that vary a combination of the at least one characteristic of the signal and an activation status of the plurality of antennas. The method also includes measuring a power of a received signal for each configuration of the multiple array configurations of the antenna array. The method further includes selecting an array configuration of the multiple array configurations based at least in part on results of the measuring and setting the antenna array to the array configuration selected. The method may also include receiving, while the antenna array is in the array configuration selected, data from at least one device chosen from among an analyte sensor on the user-body and a controller paired with the automated medicament delivery device.

In one or more illustrative embodiments, an automated medicament delivery device for automated administration of medicament to a user-body is disclosed. The automated medicament delivery device includes a delivery system, an antenna array, one or more processors, and memory. The delivery system is configured to deliver medicament to the user-body. The antenna array includes a plurality of antennas. The memory includes instructions that, when executed, cause the one or more processors to cause the antenna array to transition between multiple array configurations. The instructions, when executed, also cause the one or more processors to measure a power for each configuration of multiple array configurations of the antenna array. The multiple array configurations may be chosen from among configurations that vary at least one characteristic of a signal emitted or received by one or more antennas of the plurality of antennas, configurations that vary an activation status of the plurality of antennas, and configurations that vary a combination of at least one characteristic of a signal and the activation status of the plurality of antennas. The instructions, when executed, further cause the one or more processors to select an array configuration of the multiple array configurations based at least in part on results of the measuring and set the antenna array to the array configuration selected. The instructions, when executed, may also cause the one or more processors to receive, while in the antenna array is in the array configuration based at least in part on results of the measuring, data from at least one device chosen from among an analyte sensor on the user-body and a controller paired with the automated medicament delivery device.

### BRIEF DESCRIPTION OF THE DRAWINGS

For a detailed understanding of the disclosure, reference should be made to the following detailed description, taken in conjunction with the accompanying drawings, in which like elements have generally been designated with like numerals, and wherein:

FIG. 1 is a schematic diagram illustrating an automated medicament delivery device (or system);

FIG. 2 is a block diagram of an automated medicament delivery system for controlled administering of medicament, in accordance with one or more embodiments;

FIG. 3 is a top view of a PCB with an antenna array connected thereto, in accordance with one or more embodiments;

FIG. 4 is a perspective view of a PCB with an antenna array connected thereto, in accordance with one or more embodiments;

FIG. 5 is a schematic diagram of an embodiment of an antenna array, in accordance with one or more embodiments;

FIG. 6 is a schematic diagram of an embodiment of the antenna array, in accordance with one or more embodiments;

FIG. 7 is a schematic diagram of an embodiment of the antenna array, in accordance with one or more embodiments;

FIG. 8 is a schematic diagram of an embodiment of the antenna array, in accordance with one or more embodiments;

FIG. 9 illustrates a radiation pattern of the antenna array of FIG. 3 with a maximum gain direction steered in a first direction;

FIG. 10 illustrates a radiation pattern of the antenna array of FIG. 3 with the maximum gain direction steered in a second direction;

FIG. 11 illustrates a radiation pattern of the antenna array of FIG. 3 with the maximum gain direction steered in a third direction;

FIG. 12 illustrates the maximum gain direction being steered to reflect off of a reflective surface;

FIG. 13 illustrates the maximum gain direction being steered across a user-body;

FIG. 14 is a flowchart of a method for controlling an antenna array of an automated medicament delivery device (or system) or other wearable devices; and

FIG. 15 is a flowchart of a method for updating the configuration of the antenna array.

### DETAILED DESCRIPTION

In various embodiments, an automated medicament delivery system or device includes an antenna array configurable to control and modify a gain pattern thereof to improve communication with one or more devices (e.g., an analyte sensor and a controller paired therewith, without limitation). The antenna array includes multiple antennas and may include signal modulators, switches, or a combination thereof that are configurable for modifying the gain pattern to direct the gain pattern and/or maximize the gain pattern in a direction of the one or more devices. The antenna array may improve communication by maximizing the gain pattern in a direction along the user-body or away from the user-body to improve communication with other devices.

The illustrations presented herein are not actual views of any system, device, or structure, or any component thereof, but are merely idealized representations, which are employed to describe various embodiments.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any system, device, or structure, when utilized in a conventional manner. Furthermore, these terms may refer to an orientation of elements of any system, device, or structure, as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

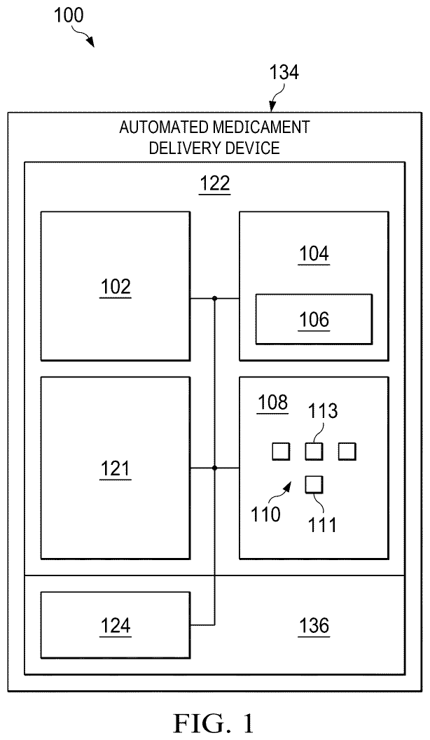

FIG. 1 is a schematic diagram illustrating an automated medicament delivery device (or system) 100 for automated administration of medicament to a user-body, in accordance with one or more embodiments.

In one or more embodiments, the automated medicament delivery device 100 may be capable of one or more operative modes of administration of medicament. Non-limiting examples of the one or more operative modes include: fully automated administration of medicament, partially automated administration of medicament, or manual administration of medicament. In one or more embodiments, the automated medicament delivery device 100 may be capable of alternating between multiple (e.g., two or more, without limitation) operative modes. As a non-limiting example, the automated medicament delivery device 100 may alternate between one or more of: fully automated operation, partially automated operation, and manual operation.

The automated medicament delivery device 100 may administer medicament at least partially based on one or more values representative of amounts of one or more analytes present within a user-body (herein after, such values respectively referred to as an “analyte value”). The one or more analytes may include constituents of the user-body and foreign substances, such as medicaments, markers, metabolites, and combinations or subcombinations of one or more of the foregoing, without limitation. The automated medicament delivery device 100 may also administer an amount of medicament at least partially based on user inputs (e.g., a user-defined bolus amount or details related to a meal consumed or about to be consumed, such as number of carbohydrates, amount of fat, and amount of protein, without limitation).

Non-limiting examples of medicaments administrable by the automated medicament delivery device 100 include: insulin, glucagon-like peptide-1 receptor agonist (GLP-1), glucose-dependent insulinotropic polypeptide (GIP), pramlintide, or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

The automated medicament delivery device 100 is configured to administer medicament to a user-body, such as subcutaneously into the user-body, without limitation, in accordance with one or more embodiments. In one or more embodiments, the automated medicament delivery device 100 may offer one or more operative modes for administration of medicament to a user-body. When operating in some of the operative modes, automated medicament delivery device 100 may administer medicament at least partially responsive to analyte values, including without limitation analyte values received from an analyte sensor. The analyte sensor is configured to obtain data related to one or more analytes within the user-body (“analyte data”). The analyte sensor may be an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G7 manufactured by Dexcom, without limitation).

When operating in some further operative modes, automated medicament delivery device 100 may administer medicament at least partially responsive to user input. When operating some yet further operative modes, automated medicament delivery device 100 may administer medicament at least partially responsive to analyte values and user input. Non-limiting examples of the one or more operative modes offered by automated medicament delivery device 100 include: fully automated administration of medicament, partially automated administration of medicament, or manual administration of medicament. When operating in an operative mode that includes manual administration of medicament, automated medicament delivery device 100 may administer medicament solely in response to a user input (e.g., delivers medicament in response to a user confirmation of delivery of medicament or in response to a user instruction to delivery medicament, without limitation). When operating in an operative mode that includes fully automated administration of medicament, automated medicament delivery device 100 may administer medicament solely in response to analyte values (e.g., delivers medicament in response to one or more analyte values, without limitation). When operating in an operative mode that includes partially automated administration of medicament, automated medicament delivery device 100 may administer medicament in response to analyte values and user input (e.g., delivers medicament in response to a user input and an analyte value, or alternately delivers medicament in response to a user input or in response to analyte values, without limitation). Medicament administration may include administration of a basal amount of medicament regularly delivered a control interval (e.g., at a determined basal rate, without limitation) to keep analyte levels stale and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user-defined bolus (e.g., responsive to user inputs provided, such as a user-defined bolus amount or details related to a meal consumed or about to be consumed, such as number of carbohydrates, amount of fat, and amount of protein, without limitation).

The automated medicament delivery device 100 includes a delivery system 124, one or more processors 102, memory 104, communication equipment 108, a PCB 122, and a power source 121. The automated medicament delivery device 100 may also include a housing 134 configured to enclose the various components of the automated medicament delivery device 100 and a chassis 136 configured to hold or support one or more components (e.g., one or more components of the delivery system 124) of the automated medicament delivery device 100. In one or more embodiments, the automated medicament delivery device 100, or portions thereof, may be a wearable device and may be secured to a user-body via an adhesive liner.

In various embodiments, the delivery system 124 is configured to cause an amount of medicament to move (e.g., flow, without limitation) toward and/or into a user-body The delivery system 124 may deliver amounts of medicament at least partially responsive to requests. In various embodiments, instructions 106 of memory 104 may include instructions for determining and generating requests for delivery system 124. In various embodiments, instructions 106 may include instructions for determining one or more amounts of medicament, determining a timing for delivery of one or more amounts of medicament, and for generating one or more requests for delivery system 124 related to the same. When such instructions of instructions 106 are executed by one or more processors 102, the one or more processors 102 determine the amounts of medicament and timing of delivery, generate requests for the delivery system 124 at least partially based on the determined amounts and timing, and provide the requests to delivery system 124.

The communication equipment 108 is configured to facilitate communication (e.g., wireless communication, without limitation) of the automated medicament delivery device 100 with other devices including, without limitation, communication between the automated medicament delivery device 100 and the analyte sensor and/or a controller (e.g., a dedicated electronic device, a smart phone, a tablet computer, a wearable device, without limitation). The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol.

The communication equipment 108 includes an antenna array 110 for the wireless communication. As will be described in further detail below, the antenna array 110 includes multiple antennas 111 spaced apart from one another. In various embodiments, the communication equipment 108 includes an Internet of Things (IOT) Subscriber Identity Module (SIM) card (e.g., a machine-to-machine SIM card, a Universal Integrated Circuit Card, without limitation). In various embodiments, the communication equipment 108 includes one or more communication chips 113 configured to control the antenna array 110 and communication via the antenna array 110. The one or more communication chips 113 may include one or more processors and memory, which may be separate from or integrated into the one or more processors 102 and the memory 104. The one or more communication chips 113 may include a Receive Signal Strength Indicator (RSSI), which may be configured to measure a power received by the antenna array 110.

The memory 104, one or more processors 102, and communication equipment 108 may be on and electrically connected via the PCB 122.

The power source 121 is configured to supply power to the delivery system 124 and the various electronic components, such as the one or more processors 102, memory 104, communication equipment 108, and the like. Power source 121 may be, as a non-limiting example, a power storage device (e.g., a battery, without limitation), a power inlet, a power regulator, or combination thereof.

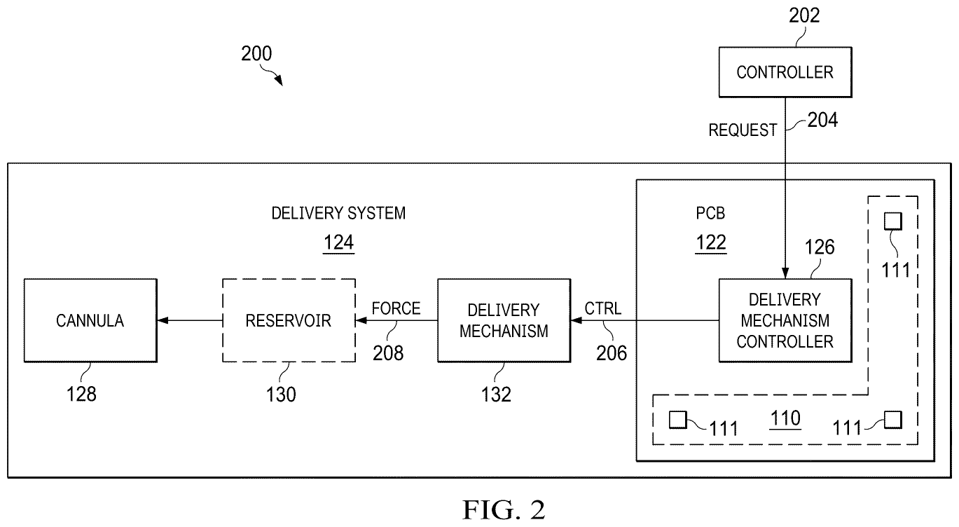

FIG. 2 is a block diagram of an automated medicament delivery system 200 for controlled administration of medicament to a user-body, in accordance with one or more embodiments.

Automated medicament delivery system 200 includes a delivery system 124 and an integrated controller 202 (“controller 202”). Delivery system 124 includes delivery mechanism controller 126, delivery mechanism 132, cannula 128, and reservoir 130. The reservoir 130, which holds the medicament, may be configured as a permanent fixture within the device or as a replaceable component, as a non-limiting example based on user needs or medicament refill practices. In FIG. 2, the block representing the reservoir 130, which stores the medicament, is outlined in dashed lines to indicate it may be either permanently within delivery system 124 or replaceable by a user within delivery system 124.

The controller 202 is configured to manage automated medicament delivery device 100 and, more generally, administration of medicament to a user-body. In one or more embodiments, controller 202 may be implemented by instructions 106 and one or more processors 102 of the automated medicament delivery device 100 of FIG. 1.

In various embodiments, controller 202 and delivery system 124 may be realized in different devices (e.g., controller 202 may be realized in a physically different device (or devices) than delivery system 124 is realized, or in the same device). When realized in different devices, functionality of controller 202 and delivery system 124 may be implemented, at least in part, by respective memory and one or more processors of their respective devices. When realized in a same device, functionality of controller 202 and delivery system 124 may be implemented, at least in part, by memory and one or more processors, respective memory and respective one or more processors, or a combination thereof. Non-limiting examples of devices in which controller 202, or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 202 may be configured to receive analyte data (e.g., from the analyte sensor via the antenna array 110, without limitation) including analyte values. In one or more embodiments, controller 202 may determine information about analytes within a user-body at least partially based on analyte data, for example, amounts, trends, distributions, without limitation. The controller 202 may analyze information about analytes in a user-body and may present the information and/or analysis to a patient, caregiver, or healthcare provider, as a non-limiting example, via an application (e.g., executing on a personal computer, smart phone, cloud server, or combinations thereof).

In various embodiments, the controller 202 may be configured to receive information from inputs from the patient or a caregiver (e.g., when the patient ate a meal or when the patient exercised, without limitation), and inputs from other electronic devices (e.g., information from a smartwatch, without limitation) and to utilize such information (e.g., process such information utilizing a control algorithm, without limitation) as discussed herein. For example, in various embodiments, controller 202 may utilize some or a totality of such information to determine amounts of medicament to administer and timing of administration of medicament. Further, controller 202 may also be configured to determine requests, including request to administer dose 204, and send those requests to the automated medicament delivery device 100.

In various embodiments, controller 202 may be configured to determine a target dose amount to administer to a user of automated medicament delivery system 200. Controller 202 may determine a target dose amount at least partially based on one or more of therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), doses of fast-acting or rapid-acting insulin, a current glucose value, and derivatives thereof without limitation. The timing and target dose amounts associated with requests generated by controller 202 may be governed by one or more control algorithms, discussed below.

Controller 202 may send a request to administer dose 204 to delivery system 124, and more specifically, delivery mechanism controller 126.

The cannula 128 is insertable into a user-body (e.g., with a tip thereof positioned subcutaneously, without limitation) and is configured to provide medicament to a user-body (e.g., subcutaneously into the user-body, without limitation).

The reservoir 130 is configured to store and retain a medicament therein. As a non-limiting example, the reservoir 130 may be a hollow body, a flexible pouch, a chamber, a vial, without limitation. In various embodiments, reservoir 130 is a fluid reservoir for holding medicament and may be, as a non-limiting example, formed from the walls of a cartridge. In the cartridge example, delivery system 124 may include a chamber (i.e., a space or region defined within delivery system 124) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 130 will be greater in a pre-filled state than the volume in an exhausted state. Additionally or alternatively to the cartridge example, delivery system 124 may be a multi-part delivery device where one of the two parts includes the reservoir 130 and the other one of the two parts includes the delivery mechanism controller 126. The other one of the two parts may optionally further include controller 202. Either one of the two parts may optionally include delivery mechanism 132 (e.g., a piston pump or a reciprocating pump, without limitation). The one of the two parts that includes reservoir 130 may be disposable (i.e., a “disposable part”) and configured to be removably secured to the other part of automated medicament delivery system 200. When reservoir 130 is exhausted, the disposable part may be removed and a replacement part including a reservoir 130 optionally in a pre-filled state, may be installed.

Delivery mechanism 132 is configured to urge fluid in reservoir 130 toward an interface for dispensing fluid (interface not shown). In various embodiments, delivery mechanism 132 may be positioned adjacent to reservoir 130. The delivery mechanism 132 is configured to cause an amount of the medicament to be administered to the user-body by causing the amount to flow from the reservoir 130 toward and into a user-body via cannula 128, which is in fluidic communication with the reservoir 130. In various embodiments, delivery mechanism 132 may utilize any suitable mechanism to generate positive displacement or negative displacement to transfer amounts of medicament from reservoir 130 toward cannula 128 and a user-body.

For example, delivery mechanism 132 may apply a force to a piston free to move within reservoir 130, and via such a force, move the piston in a direction that urges fluid in reservoir 130 toward the aforementioned interface. In one or more embodiments, delivery mechanism 132 may include an electrical motor (e.g., an AC or DC motor) that produces a force to, directly or indirectly, move the piston to perform a delivery action. A delivery action dispenses at a predetermined rate or volume of medicament (i.e., a predictable amount of fluid over a predictable duration of time). The delivery mechanism 132 may be capable of multiple rates of delivery, and in one or more embodiments, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal 206 may be determined and generated by delivery mechanism controller 126 to correspond to a delivery action. A control signal 206 may also be referred to herein as a “command 206” or an “instruction 206.”

Delivery mechanism controller 126 may generate control signal(s) 206 corresponding to one or more delivery actions at least partially based on a request to administer dose 204 received from controller 202. Control signal 206 may include first control signals to cause delivery mechanism 132 to generate resultant force 208, and a second, different control signal(s) to cause delivery mechanism 132 to not or stop generating force 208. Utilizing control signals 206, delivery mechanism controller 126 may control a length of a duration of time that delivery mechanism 132 produces force 208 and applies it to dispense fluid from reservoir 130, and indirectly, an amount of fluid dispensed from reservoir 130.

When delivery mechanism controller 126 generates control signal(s) 206 in response to a request to administer dose 204 from controller 202, it may generate the control signal(s) 206 at least partially based on a value of a target dose amount included with, or indicated by, request to administer dose 204. One or more delivery actions may be utilized to dispense an amount of fluid corresponding to a dose amount determined by controller 202. For example, a fluid amount dispensed according to a delivery action may be less than a dose amount. Generally speaking, the delivery mechanism 132 and delivery system 124 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more embodiments, delivery mechanism controller 126 may be configured to determine and generate feedback information about delivery actions, such as times of delivery actions and dispensed amounts, without limitation. Feedback information may be generated based on information generated by delivery mechanism 132 or by sensors utilized by delivery mechanism controller 126 to monitor operation of delivery mechanism 132 (sensors not depicted). For example, sensors to monitor mechanical movement, current consumption, a voltage profile of an electric motor, reservoir fluid amount, without limitation, may be utilized. Such information may be logged and provided to and stored at controller 202, without limitation, e.g., for later processing or reading, without limitation. For example, the logs can be processed to determine patterns that may be utilized to determine whether delivery system 124 is operating as expected (e.g., in a predictable manner, without limitation), and if a difference between actual and expected operation exceeds a threshold, delivery mechanism controller 126 may be updated (e.g., firmware, parameters, or both, of delivery mechanism controller 126 may be updated, without limitation) to compensate or correct for the difference. Additionally or alternatively to updating the firmware or parameters, in a multi-part system, one or more parts including delivery mechanism controller 126 or controller 202 may be indicated as needing replacement (e.g., an alarm or alert is generated at delivery system 124, automated medicament delivery system 200, a mobile device or computer in communication therewith, without limitation).

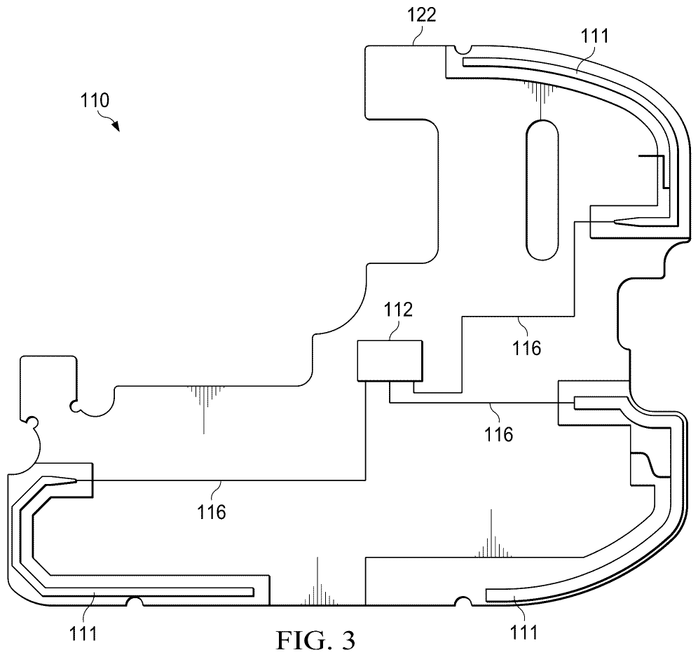

FIG. 3 is a top view of a PCB with an antenna array 110 connected thereto, in accordance with one or more embodiments. Referring to FIG. 3, antenna array 110 includes the multiple antennas 111 and a power circuit 112. The multiple antennas 111 include at least two antennas 111 and may include any number of antennas 111 (e.g., 2, 3, 4, 5, or more antennas 111, without limitation).

Each of the multiple antennas 111 may be any type of antenna usable within the automated medicament delivery device (or system) 100 or other wearable devices. Each of the multiple antennas 111 may be the same type of antenna or may be a different type of antenna. In various embodiments, at least one antenna 111 of the multiple antennas 111 is oriented with a maximum antenna field strength in a different direction than at least one other antenna 111 of the multiple antennas 111 (e.g., antennas 111 of a same type oriented in different directions, without limitation). In various embodiments, each of the multiple antennas 111 is oriented with a maximum antenna field strength in different directions than all other antennas 111 of the multiple antennas 111. In various embodiments, each of the multiple antennas 111 is positioned at or connects to a different corner of the PCB 122.

Each antenna 111 may be an on-board antenna (e.g., printed on the PCB 122, without limitation) or an off-board antenna (e.g., connected to the PCB 122 and positioned away from the PCB 122, such as being positioned within the housing 134, embedded within a wall of the housing 134, or extending out of the housing 134, without limitation). For example, an off-board antenna may include an elevation portion and an upper portion. The elevation portion may generally extend in a direction transverse to a plane defined by a surface of the PCB 122 with a connection end electrically connecting the off-board antenna to the PCB 122. The upper portion may connect to the elevation portion distal to the connection end and may generally extend transverse to the elevation portion, offset from the PCB 122. The upper portion may be secured to and/or embedded in at least one support element chosen from among the chassis 136 and the housing 134.

In various embodiments, the power circuit 112 includes a power splitter/combiner configured to feed each of the multiple antennas 111 (e.g., split a single signal into multiple outputs, without limitation) from a same chip (e.g., the one or more processors 102, without limitation) and direct/combine the signal(s) received by the multiple antennas 111 (e.g., combine multiple signals into a single output or direct a single signal from any of the multiple antennas 111 to a single output, without limitation) to the same chip (e.g., a communication chip 113 and/or the one or more processors 102, without limitation). Each of the multiple antennas 111 connects to the power circuit 112 via one or more transmission lines 116 (e.g., a radio frequency feed line, without limitation).

The antenna array 110 may also include other elements that optimize the signals of each of the multiple antennas 111 (e.g., a parasitic element physically separated from an antenna (i.e., contactless/not connected to the antenna) that is configured to function as a second resonator and couple electromagnetically to the antenna, without limitation).

FIG. 4 is a perspective view of a PCB 122 with an antenna array 110 connected thereto, in accordance with one or more embodiments. In various embodiments, the multiple antennas 111 include different types of antennas 111, such as PIFA antennas 111 configured to direct energy 118 along a user-body, patch antennas 111 configured to direct energy 118 away from the user-body, off-board antennas, and other antennas usable within the automated medicament delivery device (or system) 100 or other wearable devices.

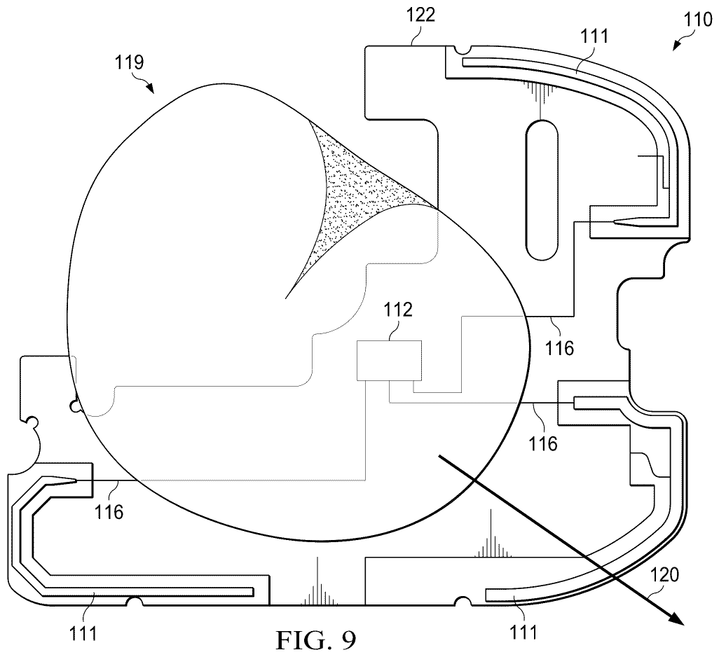

In various embodiments, each of the antennas 111 radiates a substantially similar signal simultaneously and a strength of the combined signal is shown by a resulting radiation pattern (refer to FIGS. 9-11, for example). The phase of the signal reaching each antenna 111 may be set to produce a maximum signal strength for a given direction. In various embodiments, the phase of each antenna 111 is based on a length of the transmission line 116 to fix a maximum antenna field strength of the respective antenna 111 in a different direction than the other antennas 111 and spread the signal over a wider spatial area. The phase of the signal reaching each antenna 111 may be set to spread the signal over the widest area possible and to fill dead spots. As will be discussed in further detail below, one or more of the characteristics of the signal emitted or received by one or more antennas 111 of the antenna array 110 may be adjusted to modify the direction of the maximum signal strength. The characteristics may be adjusted by modifying a path length for the signal or signal modulators (e.g., signal modulators 114, discussed below).

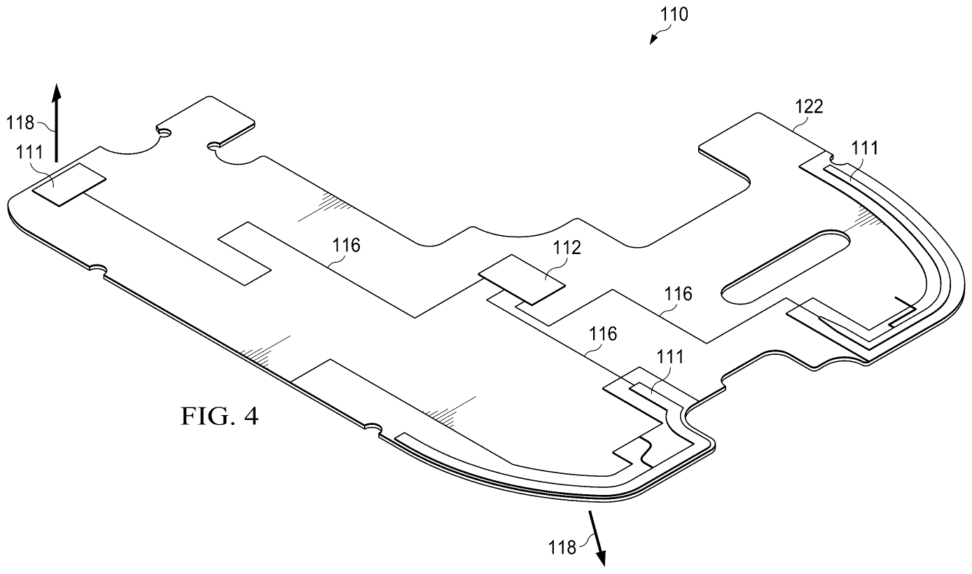

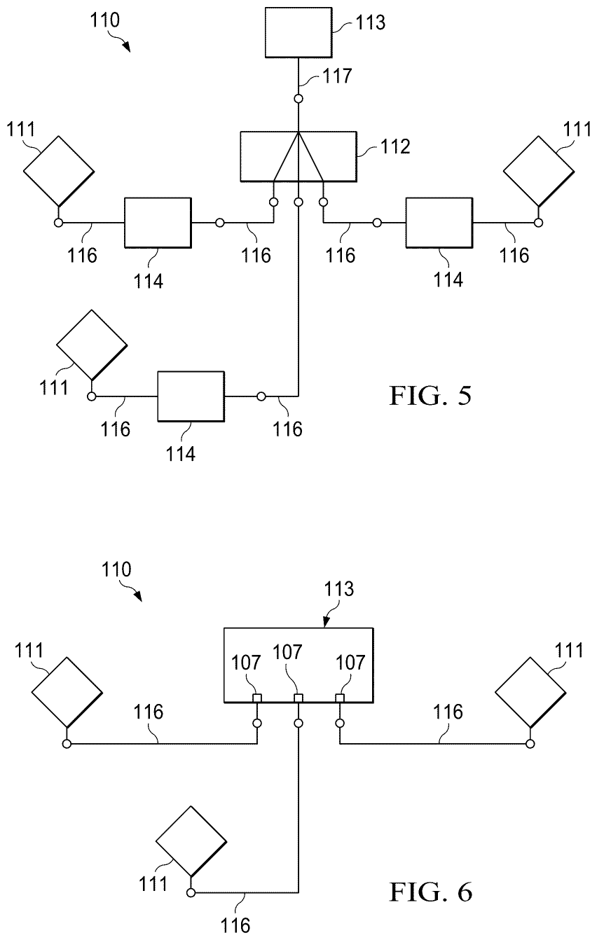

FIG. 5 is a schematic diagram of an embodiment of the antenna array 110 of FIG. 3. Referring to FIG. 5, in various embodiments, the antenna array 110 includes multiple signal modulators 114. Each signal modulator 114 corresponds to one antenna 111 of the multiple antennas 111. Each signal modulator 114 may be positioned on an antenna path between the power circuit 112 and the one antenna 111 corresponding thereto (e.g., connecting the one antenna 111 corresponding thereto to the power circuit 112 via transmission lines 116, without limitation). Each signal modulator 114 is configured to adjust at least one characteristic of a signal emitted or received by the one antenna 111 corresponding thereto. In various embodiments, the at least one characteristic is chosen from among a phase of the signal and an amplitude of the signal. In various embodiments, the at least one characteristic also includes an impedance of the signal. Each signal modulator 114 may comprise one or more of a phase shifters (e.g., switched line phase shifters, without limitation) amplitude modulators (e.g., variable gain amplifiers or switched banks of resistors, without limitation), and impedance modulators, without limitation.

The communication chip 113 may be connected to the power circuit 112 via a transmission line 117 (e.g., a trace on the PCB 122, without limitation). In various embodiments, the communication chip 113 is configured to (e.g., memory including instructions, when executed, causes one or more processors to, without limitation) control the multiple signal modulators 114 and cause the multiple signal modulators 114 to adjust the phase of the signal for each of the multiple antennas 111.

FIG. 6 is a schematic diagram of an embodiment of the antenna array 110, in accordance with one or more embodiments. Referring to FIG. 6, in various embodiments, the communication chip 113 includes the power circuit 112 and the signal modulators 114 incorporated therein via hardware, software, or a combination thereof, without limitation.

In various embodiments, the communication chip 113 includes multiple receivers 107 (e.g., at least two, without limitation). The communication chip 113 may include a receiver 107 for each antenna 111. The communication chip 113 may be configured to utilize the multiple receivers 107 to determine an angle of arrival of the signal.

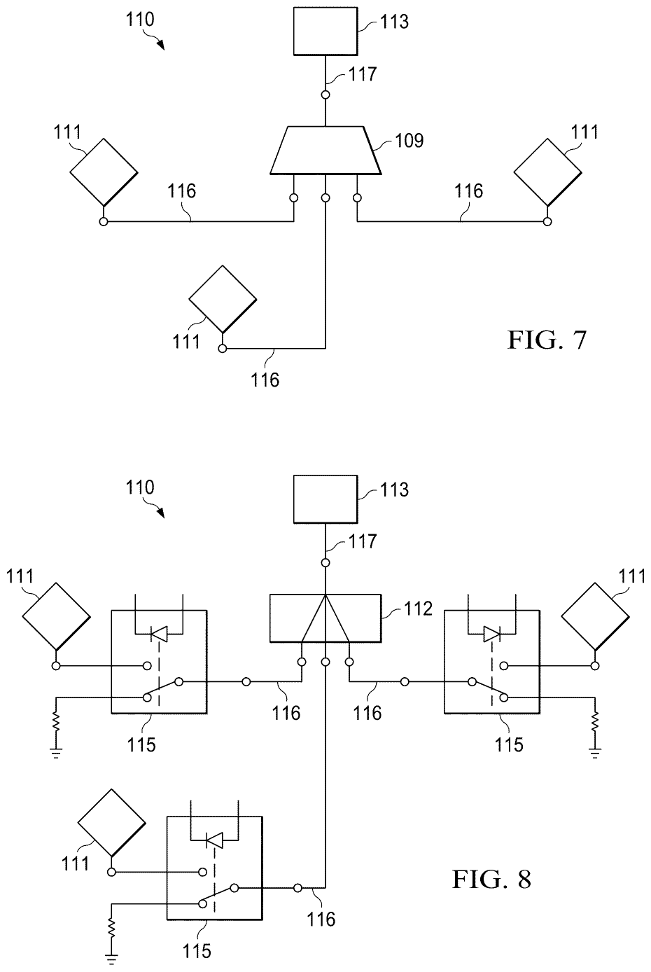

FIG. 7 is a schematic diagram of an embodiment of the antenna array 110, in accordance with one or more embodiments. Referring to FIG. 7, in various embodiments, the antenna array 110 includes a separate front-end chip 109. Referring to FIG. 7, in various embodiments, the front-end chip 109 includes any of the power circuit 112, the signal modulators 114, switches, and one or more receivers 107 incorporated therein via hardware, software, or a combination thereof, without limitation.

The signal modulators 114 and the power circuit 112 may be separate hardware elements (e.g., analog elements, without limitation), may be implemented in the communication chip 113 (e.g., via hardware, software, or a combination thereof, without limitation), or may be implemented in a separate front-end chip 109 (e.g., via hardware, software, or a combination thereof, without limitation; refer to FIG. 7, discussed below). Each signal modulator 114 may comprise one or more of a phase shifting device (e.g., switched line phase shifters, without limitation), digital phase shifters (e.g., phase control elements implemented in software, such as within the communication chip 113 or within the front-end chip 109, without limitation), amplitude modulators (e.g., variable gain amplifiers or switched banks of resistors, without limitation; refer to FIG. 5), and digital amplitude modulators (e.g., amplitude control elements implemented in software, such as within the communication chip 113 or within the front-end chip 109, without limitation).

FIG. 8 is a schematic diagram of another embodiment of the antenna array 110 of FIG. 3. Referring to FIG. 8, in various embodiments, the antenna array 110 includes multiple switches 115. Each switch 115 corresponds to one antenna 111 of the multiple antennas 111. Each switch 115 may be positioned on an antenna path between the power circuit 112 and the one antenna 111 corresponding thereto (e.g., connecting the one antenna 111 corresponding thereto to the power circuit 112 via transmission lines 116, without limitation). Each switch 115 is configured to toggle between an antenna path (e.g., a path on transmission line 116, without limitation) to activate the corresponding antenna 111 (an activated condition) and a terminating impedance to deactivate the corresponding antenna 111 (a deactivated condition). The terminating impedance may maintain a correct impedance matching seen by the communication chip 113. Each antenna 111 of the antenna array 110 may be oriented in different directions than all other antennas 111 of the multiple antennas 111.

In various embodiments, the communication chip 113 is configured to (e.g., memory including instructions, when executed, causes one or more processors to, without limitation) control the multiple switches 115 to control which of the multiple antennas 111 is actively communicating with other devices (e.g., a controller and analyte sensor, without limitation).

In various embodiments, the antenna array 110 includes multiple signal modulators 114 (refer to FIG. 5) and multiple switches 115 with one signal modulator 114 and one switch 115 corresponding to one antenna 111, each on the antenna path between the power circuit 112 and the one antenna 111. The communication chip 113 may be configured to control the multiple signal modulators 114 and causes the multiple signal modulators 114 to adjust one or more characteristics (e.g., the phase, amplitude, and/or impedance, without limitation) of the signal for each of the multiple antennas 111 and may be configured to control the multiple switches 115 to control which of the multiple antennas 111 is actively communicating with other devices (e.g., a controller and analyte sensor, without limitation). In various embodiments, signal modulators 114 are used in place of switches 115 to control which of the multiple antennas 111 is not actively communicating with other devices (e.g., by modulating the amplitude or impedance of the signal, without limitation).

In various embodiments, one or more switches 115 is configured to toggle between multiple antenna paths, each path being configured to cause a change to one or more characteristics of the signal emitted or received by the corresponding antenna 111 (multiple activated conditions). Each of the antenna paths including a different combination of one or more off a path length, amplifiers, diodes, or other electronic components that modify one or more characteristics of the corresponding antenna 111.

In various embodiments, the communication chip 113 is configured to digitally adjust one or more characteristics of the signal sent out of the communication chip 113 to adjust the one or more characteristics of the signal sent out by one or more of the antennas 111.

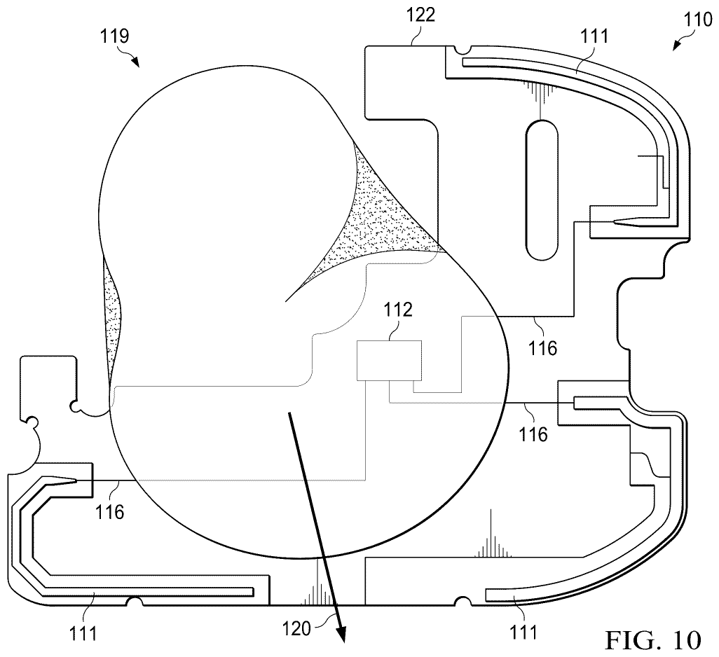

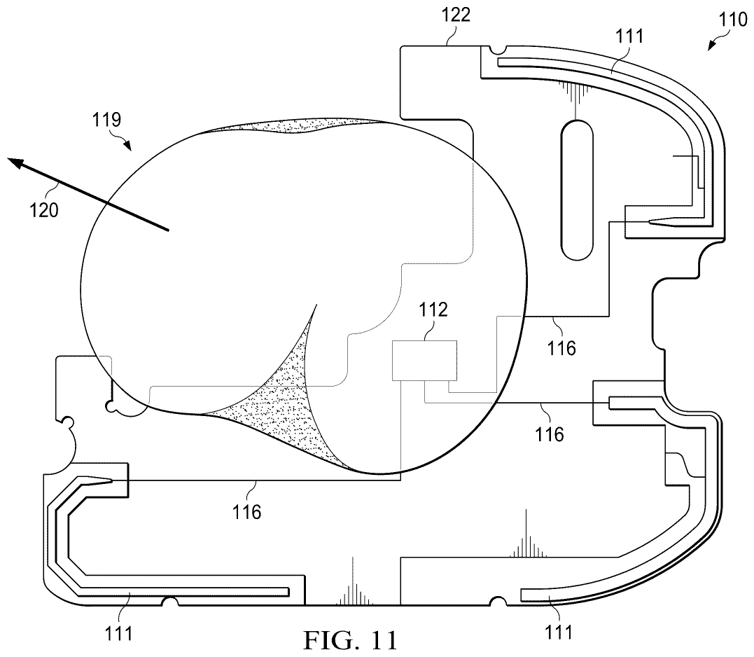

FIG. 9 illustrates a radiation pattern of the antenna array 110 of FIG. 3 with a maximized gain direction 120 steered in a first direction. FIG. 10 illustrates a radiation pattern of the antenna array 110 of FIG. 3 with the maximized gain direction 120 steered in a second direction. FIG. 11 illustrates a radiation pattern of the antenna array 110 of FIG. 3 with the maximized gain direction 120 steered in a third direction. Referring to FIGS. 9-11, in various embodiments, the one or more processors 102 are configured to adjust at least one characteristic of the signal emitted or received by one or more of the multiple antennas 111 to maximize a gain of the combined signal of the multiple antennas 111 in a particular direction (i.e., the maximized gain direction 120), which may optimize operation of the antenna array 110.

FIGS. 9-11 illustrate the antenna array gain 119 of the antenna array 110 steered to maximize the gain in the maximized gain direction 120 (in a first direction in FIG. 9, a second direction in FIG. 10, and a third direction in FIG. 11).

Maximizing the antenna array gain 119 in a maximized gain direction 120 may allow the power emitted by the antenna array 110 to be sent in a particular direction (i.e., steered) to avoid interference or to take advantage of a high-throughput channel that may randomly appear as the user moves in his environment. Further, a signal strength between the automated medicament delivery device (or system) 100 and an analyte sensor may not depend on an orientation of the automated medicament delivery device (or system) 100 as the maximized gain direction 120 for communicating with the analyte sensor may be directed at an angle toward the analyte sensor. The user can place the automated medicament delivery device (or system) 100 in the most comfortable position and orientation, and the signal therefrom can be steered in the direction of the analyte sensor.

Further, the antenna array 110 may be steered away from interference (e.g., a “noisy” environment with many other users, without limitation) and toward the intended signal. The antenna array 110 may also be steered and modified when entering new electromagnetic environments as the user moves from place to place.

By combining multiple antennas 111 into the antenna array 110, the gain of the antenna array gain 119 in the maximized gain direction 120 increases (e.g., in some configurations of the antenna array 110, the gain scales according to 10*log₁₀(N), where N is the number of antennas, without limitation). Further, the effective communication range and overall reliability of the communication between automated medicament delivery device (or system) 100 with other devices may be increased.

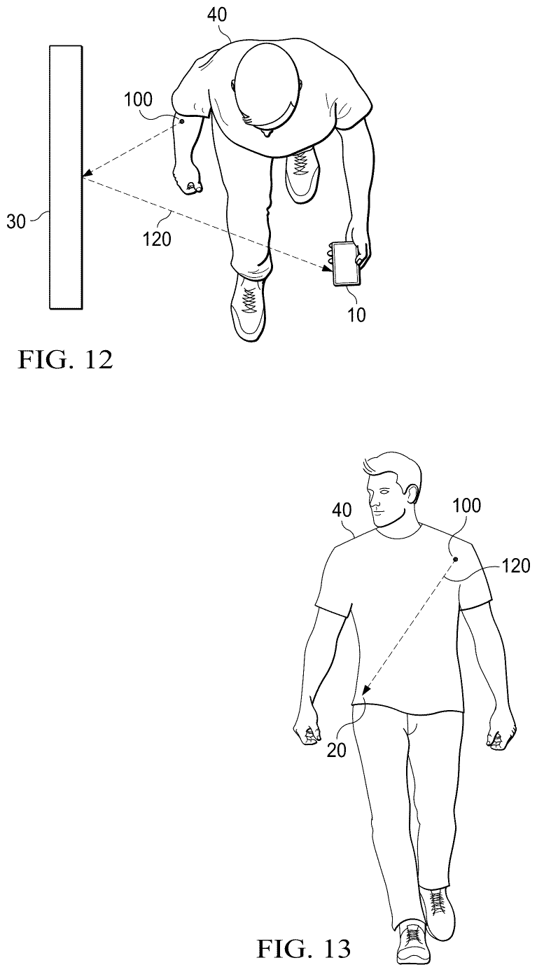

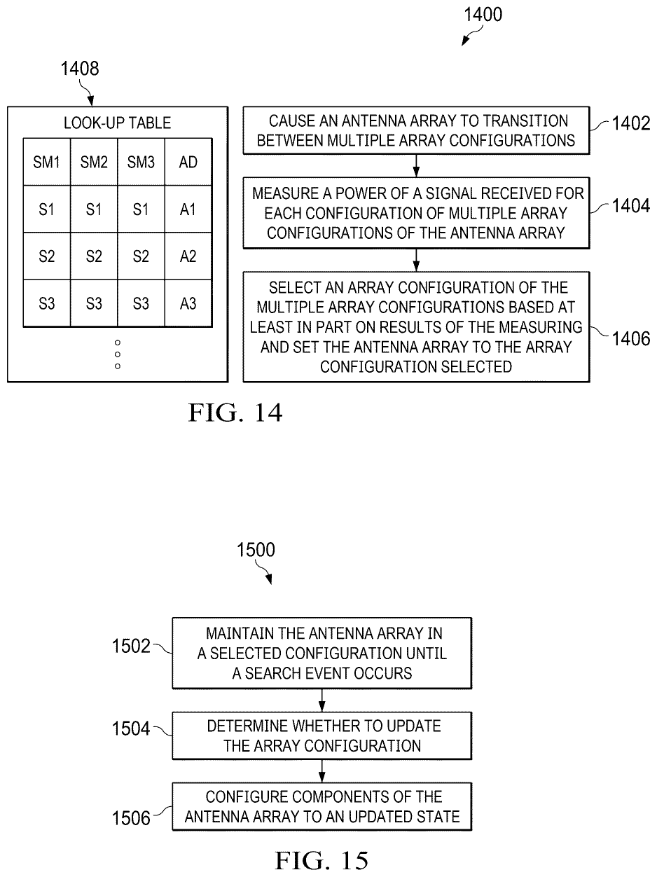

FIG. 12 illustrates the maximized gain direction 120 being steered to reflect off of a reflective surface 30. FIG. 13 illustrates the maximized gain direction 120 being steered across a user-body 40. Referring to FIGS. 9 and 10, as noted above, the antenna array gain 119 may be maximized in a maximized gain direction 120 to steer the signal in a direction that optimizes communication of the automated medicament delivery device (or system) 100 with other devices such as an analyte sensor 20 and a controller 10 (e.g., a dedicated electronic device, a smart phone, a tablet computer, a wearable device, without limitation). Thus, for example, in both FIG. 12 and FIG. 13 the maximized gain direction 120 may be steered such that an analyte sensor 20 worn by a user on one portion of the user's body may be more likely to receive a communication from the automated medicament delivery device 100 worn by the user on another portion of the user's body. Both the analyte sensor 20 and the automated medicament delivery device 100 are typically configured to be worn by the user for a number of days. Thus, in one example, the automated medicament delivery device 100 may cycle through a number of different gain directions 120 when sending communications to an analyte sensor 20 and/or a controller 10, and receive feedback from the analyte sensor 20 (or controller 10) indicative of which communications were received successfully and/or the power of the signals received. In this manner, the automated medicament delivery device (or system) 100 may determine which is the maximized gain direction 120 for communicating with the analyte sensor and/or controller 10. The automated medicament delivery device 100 may continue to send communications to the analyte sensor 20 using the same maximized gain direction 120 for a duration that the analyte sensor 20 remains on the same location of the user's body, and the analyte sensor 20 or controller 10 may communicate with the medicament delivery device 100 when the analyte sensor 20 is replaced and a new sensor applied to the user's body. Alternatively, the automated medicament delivery device 100 may continue to test different gain directions 120 when sending further communications to the analyte sensor 20 or the controller 10. An example of how the automated medicament delivery device 100 may cycle through a number of different gain directions 120 is described below with reference to FIG. 14.

FIG. 12 illustrates an example where a user is in close proximity to a reflective surface 30 and the antenna array gain 119 is maximized in a maximized gain direction 120 at the reflective surface 30, which allows the signal to be reflected off of the reflective surface 30 (around the user-body 40) to optimize communication with the controller 10 positioned at an opposite side of the user-body 40.

FIG. 13 illustrates an example where the user is in an open area and the antenna array gain 119 is maximized in a maximized gain direction corresponding to the direction of the analyte sensor 20 across the user-body 40. In a test comparing the gain of a single antenna versus the gain of a three element antenna array, with a phase shift steering the signal of the three element antenna array across the user-body, the power received was increased by 4.6 dB, with a similar increase occurring for a transmit operation, illustrating that more power can be transmitted and received with an antenna array 110 compared to using just a single antenna.

FIG. 14 is a flowchart of a method 1400 for controlling an antenna array of an automated medicament delivery device (or system) or other wearable devices. The method 1400 includes causing an antenna array to transition between multiple array configurations at act 1402. The multiple array configurations may be chosen from among any of the array configurations disclosed herein or other similar array configurations, without limitation. The multiple array configurations may be chosen from among configurations that vary at least one characteristic of a signal emitted or received by one or more antennas of the plurality of antennas, configurations that vary an activation status of the plurality of antennas (e.g., via open/closed switches or a minimized amplitude of the signal, without limitation), and configurations that vary a combination of at least one characteristic of a signal emitted or received by the one or more antennas and an activation status of the plurality of antennas.

In various embodiments, the antenna array includes signal modulators, each signal modulator corresponding to an antenna of the antenna array and is configured to modify at least one characteristic of a signal emitted or received by the corresponding antenna.

A look-up table 1408 may define an antenna array configuration for each array direction AD (e.g., a first angle A1, a second angle A2, a third angle A3, . . . , an n^{th} angle, without limitation) of a predetermined number of array directions AD. Each configuration may include a shift state (e.g., S1, S2, S3, . . . , Sn^{th}, without limitation) for multiple signal modulators (e.g., SM1, SM2, SM3, . . . , SMn^{th}, without limitation) and may include a shift state for each signal modulator. Each shift state of each signal modulator may define a particular state for one or more characteristics of the signal (e.g., one or more of a particular phase and a particular amplitude of the signal, without limitation) to maximize the gain of the combined signal of the antenna array at the chosen array direction AD. In various embodiments, act 1202 includes setting each signal modulator to a defined shift state for a respective array configuration at each transition.

The look-up table 1408 may include any amount of a predetermined number of antenna array configurations. Each configuration may include an array direction AD with a unique angle corresponding thereto and corresponding shift states for one or more of the signal modulators to maximize the gain of the antenna array at the unique angle. The look-up table 1208 may be stored in memory of the automated medicament delivery device (e.g., memory of a communication chip 113 and/or memory 104, without limitation).

The multiple array configurations may include all of the predetermined number of array directions AD (e.g., the received power of all of the array directions are cycled through and measured, without limitation) in the look-up table 1408 or may include a subset of the predetermined number of array directions AD (e.g., a select number of the predetermined number of array directions are cycled through and measured, without limitation) in the look-up table 1208.

In various embodiments, the antenna array includes a switch corresponding to one or more antennas that are configured to toggle between an activated condition to activate the corresponding antenna and a deactivated condition to deactivate the corresponding antenna. In various embodiments, the shift state for one or more antenna array configurations includes a minimized amplitude for toggling an antenna to the deactivated condition and a respective amplitude, above the minimized amplitude, for toggling the antenna to the activated condition. In various embodiments, act 1402 includes setting each antenna to a defined condition chosen from the activated condition and the deactivated condition for a respective array configuration at each transition (e.g., via switches, signal modulators configured to modulate the amplitude of the corresponding signal, or impedance modulators, without limitation).

Each configuration may include a combination of activated antennas (e.g., a switch for the corresponding antenna is toggled to the antenna path for the activated condition, without limitation) and deactivated antennas (e.g., a switch for the corresponding antenna is toggled to the deactivated condition with a terminating impedance, without limitation). Each combination of activated antennas may be a unique combination.

In various embodiments, the multiple array configurations include each available configuration of a single activated antenna with all other antennas deactivated. For example, in a three antenna array, a first configuration includes a first antenna activated while a second antenna and a third antenna are deactivated, a second configuration includes the second antenna activated while the first antenna and the third antenna are deactivated, and a third configuration includes the third antenna activated while the first antenna and the second antenna are deactivated.

In various embodiments, the multiple activation configurations include each configuration with a distinct combination of one or more activated antennas (e.g., each combination of a single antenna activated, each combination of two antennas activated, and the combination of all antennas activated, without limitation).

In various embodiments, the antenna array includes multiple signal modulators (refer to FIGS. 5-7) and multiple switches (refer to FIG. 8) with each signal modulator and each switch corresponding to an antenna, each on the respective antenna path between the power circuit and the respective antenna. In various embodiments, each antenna of the antenna array includes a corresponding signal modulator and a corresponding switch. In various embodiments, the switch may include multiple activated conditions and is configured to toggle between the multiple activated conditions and the deactivated condition. Each of the multiple activated conditions may cause a length of the antenna path between the antenna and the communication chip to change, defining multiple phase conditions for the antenna.

Each configuration of the multiple array configurations may include a combination of shift states for the signal modulators and activation states for the antennas. The look-up table 1408 may include the shift states and activation states for each configuration of the predetermined number of antenna array configurations. In various embodiments, signal modulators corresponding to antennas with a corresponding switch with an activation state specified as a deactivated condition may not have a shift state specified within the look-up table 1408 or may have a shift state specified that does not apply a shift to one or more characteristics of the signal for the corresponding antenna.

The array direction may include a gain of the antenna array substantially maximized at a unique angle. A gain of the antenna array at the unique angle may be about the same with one or more of the antennas set in the deactivated condition (e.g., a maximum gain of the antenna array with the one or more of the antennas set in the deactivated condition is within a predetermined threshold value of the maximum gain of the antenna array with all of the antennas activated at the unique angle, without limitation). In this case, the gain of the antenna array may be substantially maximized at the unique angle utilizing the configuration with the one or more of the antennas set in the deactivated condition, which may conserve power consumption.

The method also includes measuring a power of a received signal of the antenna array for each configuration of multiple array configurations of the antenna array at act 1404. Act 1402 may include setting each component into a respective condition for one of the multiple array configurations prior to measuring the power of the signal received.

The power of the received signal may be measured using a receive single strength indicator (RSSI) of a communication chip (e.g., a BLUETOOTH® communication chip, without limitation) and may be power received from a paired device (e.g., a controller or analyte sensor paired with the automated medicament delivery device (or system), without limitation). In a transmit mode, a test signal may be requested and sent back to the automated medicament delivery device (or system) to determine the received power of each path array direction AD.

The method 1400 also includes selecting an array configuration of the multiple array configurations based at least in part on results of the measuring and setting the antenna array to the array configuration selected at act 1406. Act 1406 may include setting each signal modulator of the antenna array to a shift state corresponding to the configuration with the highest received power. Act 1404 may include storing the received power of the antenna array for each of the multiple array configurations measured and selecting the highest value stored. In various embodiments, the method 1400 includes periodically selecting which of the multiple array configurations results in the highest received power and maintaining a history of the configurations selected. In various embodiments, act 1404 includes selecting the array configuration with the highest power measured.

In various embodiments, selecting an array configuration of the multiple array configurations is based at least in part on energy consumed by the configuration. By considering both the power of the signal received and the energy consumed by the antenna configuration, the configuration may be selected to optimize both signal strength of the antenna array and battery life of the automated medicament device. In various embodiments, the configuration chosen substantially maximizes the power of the signal received. Substantially maximizing power may be a configuration with a measured power that is within a predetermined threshold value of the maximum power of the signal measured, without limitation. The power of the received signal may be about the same with one or more of the antennas set in a deactivated condition, and the power may be substantially maximized by selecting such a configuration to reduce energy consumption.

In various embodiments, the method 1400 includes determining an angle of arrival of a signal from a paired device is being received from, selecting the configurations with angles of the array direction that are within a predetermined range of an angle of the signal received, and performing act 1402 and act 1404 using the multiple configurations selected. The angle of arrival may be determined by utilizing multiple receivers for the antennas within either the communication chip or a separate front-end chip.

In various embodiments, the method 1400 includes determining which direction a signal from a device (e.g., a paired device, an analyte sensor, or a controller, without limitation) is being received from, selecting the multiple array configurations from the predetermined number of array configurations defined in the look-up table 1408 with an angle of the array direction closest to an angle of the signal received, and performing act 1402 and act 1404 using the multiple array configurations selected.

The method 1400 may include receiving, while the antenna array is in the array configuration with the highest power received, data from at least one device chosen from among an analyte sensor on the user-body and a controller paired with the automated medicament delivery device. The data may be chosen from analyte values from the analyte sensor and control information from the controller, and method 1400 may include causing administration of medicament at least partially responsive to the data.

FIG. 15 is a flowchart of a method 1500 for updating the configuration of the antenna array. The method 1500 may be performed after an initial selection of configuration of the antenna array utilizing the method 1400. The method 1500 includes maintaining the antenna array in a selected configuration until a search event occurs at act 1502. A search event may be any event that triggers an action for determining whether to update the configuration of the antenna array. The search event may be an occurrence of at least one event chosen from among a passing of a predetermined amount of time (e.g., re-selection of the configuration may be cyclical and recalibrated at each cycle, such as 5 minutes or 10 minutes, without limitation), a predetermined number of missed packets within a predetermined amount of time, a predetermined number of consecutively missed packets, a predetermined degradation of the received power, receipt of a communication from a different direction by a predetermined amount, initiation of communication with a different device, pairing with a new device (such as a new analyte sensor 20), and a pairing process with a new device is initiated (such as with a new analyte sensor 20), without limitation.

The method 1500 also includes determining whether to update the array configuration at act 1504. Act 1504 may include comparing the received power to a previously received power and, in response to the received power being below a threshold relative to a previous received power, determining to update the configuration of the antenna array. Act 1504 may include performing acts 1402 and 1404 of the method 1400.

The method 1500 further includes configuring components of the antenna array to an updated state at act 1506. The components of the antenna array may include signal modulators, switches, or a combination of signal modulators and switches.

In various embodiments, the automated medicament delivery device (or system) may be paired with multiple devices for communication therewith (e.g., a controller and an analyte sensor, without limitation). A search event at act 1502 may be triggered by communication with a second device after previously communicating with a first device. The method 1200 may be performed for communicating with each of the paired devices, and act 1504 may include switching the configuration stored for the second wearable device for communication therewith. In some of these various embodiments, communication with the paired devices is performed on a predetermined interval and the configuration of the antenna array is switched to a stored configuration for communication with a respective paired device prior to an occurrence of the predetermined interval (e.g., switching a predetermined amount of time prior to the predetermined interval, without limitation). For example, a first configuration for communicating with a controller and a second configuration for communicating with an analyte sensor may be stored in memory. The communication chip may be configured to switch to the first configuration prior to communicating with the controller (e.g., switch to the first configuration immediately prior to the interval for communication with the controller occurs, without limitation) and to switch to the second configuration prior to communicating with the analyte sensor (e.g., switch to the second configuration immediately prior to the interval for communicating with the analyte sensor occurs, without limitation).

In various embodiments, the method 1500 includes storing in memory one or more of the most recent configurations along with the received power measured therewith, and act 1504 includes remeasuring the received power for the most recent configuration in order of previous use, stopping in response to a remeasured received power being within a predetermined threshold of a previously measured received power (e.g., the remeasured received power being substantially the same as the previously measured received power, without limitation), and setting the antenna array to the configuration corresponding thereto.

While embodiments of the antenna array and methods for configuring the antenna array discussed herein are described in connection with automated medicament delivery systems and devices, such antenna arrays and methods for configuring the antenna arrays may also be utilized within other wearable devices (e.g., analyte sensors and controllers, without limitation) optionally with modifications that would be apparent to a person having ordinary skill in the art.

Non-limiting illustrative embodiments of this disclosure may include:

Embodiment 1: A method for controlling an antenna array of an automated medicament delivery device configured to administer medicament to a user-body, the method comprising: causing the antenna array to transition between multiple array configurations, the antenna array comprising a plurality of antennas and the multiple array configurations chosen from among configurations that vary at least one characteristic of a signal emitted or received by one or more antennas of the plurality of antennas, configurations that vary an activation status of the plurality of antennas, and configurations that vary a combination of the at least one characteristic of a signal and the activation status of the plurality of antennas; measuring a power of a received signal for each configuration of the multiple array configurations of the antenna array; selecting an array configuration of the multiple array configurations based at least in part on results of the measuring and setting the antenna array to the array configuration selected; and receiving, while the antenna array is in the array configuration selected, data from at least one device chosen from among an analyte sensor on the user-body and a controller paired with the automated medicament delivery device.

Embodiment 2: The method according to Embodiment 1, wherein causing an antenna array to transition between multiple array configurations includes setting each signal modulator of a plurality of signal modulators of the antenna array to a defined shift state for a respective array configuration at each transition, each of the plurality of signal modulators corresponding to a respective antenna of the plurality of antennas, the defined shift state setting the at least one characteristic of the signal, the at least one characteristic of the signal chosen from among a phase of the signal and an amplitude of the signal.

Embodiment 3: The method according to Embodiments 1 and 2, wherein causing an antenna array to transition between multiple array configurations includes setting each antenna of the antenna array to a defined condition chosen from an activated condition and a deactivated condition for a respective array configuration at each transition.

Embodiment 4: The method according to Embodiments 1 to 3, wherein each of the multiple array configurations includes an array direction, the array direction including a gain of the antenna array substantially maximized at a unique angle.

Embodiment 5: The method according to Embodiments 1 to 4, wherein measuring the power of the received signal for each configuration of the multiple array configurations of the antenna array includes measuring the power of the received signal for each of the multiple array configurations while communicating with the analyte sensor and measuring the received power for each of the multiple array configurations while communicating with controller; and wherein selecting the array configuration of the multiple array configurations includes selecting a first array configuration while communicating with the analyte sensor and selecting a second array configuration while communicating with the controller.

Embodiment 6: The method according to Embodiments 1 to 5, wherein setting the antenna array to the array configuration selected includes setting the antenna array to the first array configuration prior to communicating with the analyte sensor and setting the antenna array to the second array configuration prior to communicating with the controller.

Embodiment 7: The method according to Embodiments 1 to 6, further comprising determining from which direction the received signal from a device is coming from and selecting the multiple array configurations from a predetermined number of array configurations defined in a look-up table.

Embodiment 8: The method according to Embodiments 1 to 7, wherein the multiple array configurations include array directions with corresponding angles closer to the direction of the received signal than the remaining array configurations of the predetermined number of array configurations defined in the look-up table.

Embodiment 9: The method according to Embodiments 1 to 8, further comprising: maintaining the antenna array in a selected array configuration until a search event occurs, the search event chosen from among a passing of a predetermined amount of time, a predetermined number of missed packets within a predetermined amount of time, a predetermined number of consecutively missed packets, a predetermined degradation of the received power, receipt of a communication from a different direction by a predetermined amount, initiation of communication with a different device, pairing with a new device, and a pairing process with a new device is initiated; determining whether to update the array configuration including comparing a power of a currently received signal to a power of a previously received signal; and configuring components of the antenna array to an updated state in response to determining to update the array configuration.

Embodiment 10: The method according to Embodiments 1 to 9, wherein selecting an array configuration of the multiple array configurations is based at least in part on the an energy consumed by the configuration.

Embodiment 11: An automated medicament delivery device for automated administration of medicament to a user-body, the automated medicament delivery device comprising: a delivery system configured to deliver medicament to the user-body; an antenna array comprising a plurality of antennas; one or more processors; and memory including instructions, when executed, cause the one or more processors to: cause the antenna array to transition between multiple array configurations; measure a power of a received signal for each configuration of multiple array configurations of the antenna array, the multiple array configurations chosen from among configurations that vary at least one characteristic of a signal emitted or received by one or more antennas of the plurality of antennas, configurations that vary an activation status of the plurality of antennas, and configurations that vary a combination of the at least one characteristic of a signal and an activation status of the plurality of antennas; select an array configuration of the multiple array configurations based at least in part on results of the measuring and set the antenna array to the array configuration selected; and receive, while the antenna array is in the array configuration selected, data from at least one device chosen from among an analyte sensor on the user-body and a controller paired with the automated medicament delivery device.

Embodiment 12: The automated medicament delivery device according to Embodiment 11, wherein the antenna array includes a plurality of signal modulators, each signal modulator of the plurality of signal modulators corresponding to a respective antenna of the plurality of antennas, and wherein the instructions, when executed, cause the one or more processors to set each of the plurality of signal modulators to a shift state for a respective array configuration at each transition, the shift state setting the at least one characteristic of the signal, the at least one characteristic of the signal chosen from among a phase of the signal and an amplitude of the signal.

Embodiment 13: The automated medicament delivery device according to Embodiments 11 and 12, wherein the antenna array includes a plurality of switches, each switch of the plurality of switches corresponding to a respective antenna of the plurality of antennas, and wherein the instructions, when executed, cause the one or more processors to set each of the plurality of switches to a defined condition chosen from an activated condition and a deactivated condition for a respective array configuration at each transition.

Embodiment 14: The automated medicament delivery device according to Embodiments 11 to 13, wherein each of the multiple array configurations includes an array direction, the array direction including a gain of the antenna array substantially maximized at a unique angle.

Embodiment 15: The automated medicament delivery device according to Embodiments 11 to 14, wherein measuring the power of the received signal for each configuration of the multiple array configurations of the antenna array includes measuring the power of the received signal for each of the multiple array configurations while communicating with the analyte sensor and measuring the power of the received signal for each of the multiple array configurations while communicating with controller; and wherein selecting the array configuration of the multiple array configurations includes selecting a first array configuration while communicating with the analyte sensor and selecting a second array configuration while communicating with the controller.

Embodiment 16: The automated medicament delivery device according to Embodiments 11 to 15, wherein setting the antenna array to the array configuration selected includes setting the antenna array to the first array configuration prior to communicating with the analyte sensor and setting the antenna array to the second array configuration prior to communicating with the controller.

Embodiment 17: The automated medicament delivery device according to Embodiments 11 to 16, wherein the multiple array configurations include array directions with corresponding angles closer to the direction of the received signal than the remaining array configurations of the predetermined number of array configurations defined in the look-up table.

Embodiment 18: The automated medicament delivery device according to Embodiments 11 to 17, wherein the multiple array configurations include array directions with corresponding angles closer to the direction of the received signal than the remaining array configurations of the predetermined number of array configurations defined in the look-up table.

Embodiment 19: The automated medicament delivery device according to Embodiments 11 to 18, wherein the instructions, when executed, cause one or more processors to: maintain the antenna array in a selected array configuration until a search event occurs, the search event chosen from among a passing of a predetermined amount of time, a predetermined number of missed packets within a predetermined amount of time, a predetermined number of consecutively missed packets, a predetermined degradation of the received power, receipt of a communication from a different direction by a predetermined amount, initiation of communication with a different device, pairing with a new device, and a pairing process with a new device is initiated; determine whether to update the array configuration including comparing a power of a currently received signal to a power of a previously received signal; and configure components of the antenna array to an updated state in response to determining to update the array configuration.

Embodiment 20: The automated medicament delivery device according to Embodiments 11 to 19, wherein selecting an array configuration of the multiple array configurations is based at least in part on the an energy consumed by the configuration.

The embodiments described above and illustrated in the accompanying drawings do not limit the scope of the disclosure, which is encompassed by the scope of the appended claims and their legal equivalents. Any equivalent embodiments are within the scope of this disclosure. Indeed, various modifications, in addition to those shown and described herein, such as alternate useful combinations of the elements described, will become apparent to those skilled in the art from the description. Such modifications and embodiments also fall within the scope of the appended claims and equivalents.

What is claimed is:

1. A method for controlling an antenna array of an automated medicament delivery device configured to administer medicament to a user-body, the method comprising:
causing the antenna array to transition between multiple array configurations, the antenna array comprising a plurality of antennas and the multiple array configurations chosen from among configurations that vary at least one characteristic of a signal emitted or received by one or more antennas of the plurality of antennas, configurations that vary an activation status of the plurality of antennas, and configurations that vary a combination of the at least one characteristic of the signal and the activation status of the plurality of antennas;
measuring a power of a received signal for each configuration of the multiple array configurations of the antenna array;
selecting an array configuration of the multiple array configurations based at least in part on results of the measuring and setting the antenna array to the array configuration selected; and
receiving, while the antenna array is in the array configuration selected, data from at least one device chosen from among an analyte sensor on the user-body and a controller paired with the automated medicament delivery device.

2. The method of claim 1, wherein causing an antenna array to transition between multiple array configurations includes setting each signal modulator of a plurality of signal modulators of the antenna array to a defined shift state for a respective array configuration at each transition, each of the plurality of signal modulators corresponding to a respective antenna of the plurality of antennas, the defined shift state setting the at least one characteristic of the signal, the at least one characteristic of the signal chosen from among a phase of the signal and an amplitude of the signal.

3. The method of claim 1, wherein causing an antenna array to transition between multiple array configurations includes setting each antenna of the antenna array to a defined condition chosen from an activated condition and a deactivated condition for a respective array configuration at each transition.

4. The method of claim 1, wherein each of the multiple array configurations includes an array direction, the array direction including a gain of the antenna array substantially maximized at a unique angle.

5. The method of claim 1, wherein measuring the power of the received signal for each configuration of the multiple array configurations of the antenna array includes measuring the power of the received signal for each of the multiple array configurations while communicating with the analyte sensor and measuring the power of the received signal for each of the multiple array configurations while communicating with controller; and
wherein selecting the array configuration of the multiple array configurations includes selecting a first array configuration while communicating with the analyte sensor and selecting a second array configuration while communicating with the controller.

6. The method of claim 5, wherein setting the antenna array to the array configuration selected includes setting the antenna array to the first array configuration prior to communicating with the analyte sensor and setting the antenna array to the second array configuration prior to communicating with the controller.

7. The method of claim 1, further comprising determining from which direction the received signal from a device is coming from and selecting the multiple array configurations from a predetermined number of array configurations defined in a look-up table.

8. The method of claim 7, wherein the multiple array configurations include array directions with corresponding angles closer to the direction of the received signal than the remaining array configurations of the predetermined number of array configurations defined in the look-up table.

9. The method of claim 1, further comprising:
maintaining the antenna array in a selected array configuration until a search event occurs, the search event chosen from among a passing of a predetermined amount of time, a predetermined number of missed packets within a predetermined amount of time, a predetermined number of consecutively missed packets, a predetermined degradation of the received power, receipt of a communication from a different direction by a predetermined amount, initiation of communication with a different device, pairing with a new device, and a pairing process with a new device is initiated;
determining whether to update the array configuration including comparing a power of a currently received signal to a power of a previously received signal; and
configuring components of the antenna array to an updated state in response to determining to update the array configuration.

10. The method of claim 1, wherein selecting an array configuration of the multiple array configurations is based at least in part on the an energy consumed by the configuration.

11. An automated medicament delivery device for automated administration of medicament to a user-body, the automated medicament delivery device comprising:
a delivery system configured to deliver medicament to the user-body;
an antenna array comprising a plurality of antennas;
one or more processors; and
memory including instructions, when executed, cause the one or more processors to:
cause the antenna array to transition between multiple array configurations;
measure a power of a received signal for each configuration of multiple array configurations of the antenna array, the multiple array configurations chosen from among configurations that vary at least one characteristic of a signal emitted or received by one or more antennas of the plurality of antennas, configurations that vary an activation status of the plurality of antennas, and configurations that vary a combination of the at least one characteristic of the signal and the activation status of the plurality of antennas;
select an array configuration of the multiple array configurations based at least in part on results of the measuring and set the antenna array to the array configuration selected; and
receive, while the antenna array is in the array configuration selected, data from at least one device chosen from among an analyte sensor on the user-body and a controller paired with the automated medicament delivery device.

12. The automated medicament delivery device of claim 11, wherein the antenna array includes a plurality of signal modulators, each signal modulator of the plurality of signal modulators corresponding to a respective antenna of the plurality of antennas, and wherein the instructions, when executed, cause the one or more processors to set each of the plurality of signal modulators to a shift state for a respective array configuration at each transition, the shift state setting the at least one characteristic of the signal, the at least one characteristic of the signal chosen from among a phase of the signal and an amplitude of the signal.

13. The automated medicament delivery device of claim 11, wherein the antenna array includes a plurality of switches, each switch of the plurality of switches corresponding to a respective antenna of the plurality of antennas, and wherein the instructions, when executed, cause the one or more processors to set each of the plurality of switches to a defined condition chosen from an activated condition and a deactivated condition for a respective array configuration at each transition.

14. The automated medicament delivery device of claim 11, wherein each of the multiple array configurations includes an array direction, the array direction including a gain of the antenna array substantially maximized at a unique angle.

15. The automated medicament delivery device of claim 11, wherein measuring the power of the received signal for each configuration of the multiple array configurations of the antenna array includes measuring the power of the received signal for each of the multiple array configurations while communicating with the analyte sensor and measuring the power of the received signal for each of the multiple array configurations while communicating with controller; and
wherein selecting the array configuration of the multiple array configurations includes selecting a first array configuration while communicating with the analyte sensor and selecting a second array configuration while communicating with the controller.

16. The automated medicament delivery device of claim 15, wherein setting the antenna array to the array configuration selected includes setting the antenna array to the first array configuration prior to communicating with the analyte sensor and setting the antenna array to the second array configuration prior to communicating with the controller.

17. The automated medicament delivery device of claim 11, wherein the multiple array configurations include array directions with corresponding angles closer to the direction of the received signal than the remaining array configurations of a predetermined number of array configurations defined in a look-up table.

18. The automated medicament delivery device of claim 17, wherein the multiple array configurations include array directions with corresponding angles closer to the direction of the received signal than the remaining array configurations of the predetermined number of array configurations defined in the look-up table.

19. The automated medicament delivery device of claim 11, wherein the instructions, when executed, cause one or more processors to:
maintain the antenna array in a selected array configuration until a search event occurs, the search event chosen from among a passing of a predetermined amount of time, a predetermined number of missed packets within a predetermined amount of time, a predetermined number of consecutively missed packets, a predetermined degradation of the received power, receipt of a communication from a different direction by a predetermined amount, initiation of communication with a different device, pairing with a new device, and a pairing process with a new device is initiated;
determine whether to update the array configuration including comparing a power of a currently received signal to a power of a previously received signal; and
configure components of the antenna array to an updated state in response to determining to update the array configuration.

20. The automated medicament delivery device of claim 11, wherein selecting an array configuration of the multiple array configurations is based at least in part on the an energy consumed by the configuration.
